(19) United States
(12) Patent Application Publication
(10) Pub. No.: US 2020/0306508 A1
(43) Pub. Date: Oct. 1, 2020

(54) GUIDEWIRE, A METHOD FOR MANUFACTURING A GUIDEWIRE, AND A CONNECTOR PROVIDED IN A GUIDEWIRE

(71) Applicant: Molex, LLC, Lisle, IL (US)

(72) Inventor: Naoyoshi TAMURA, Yamato (JP)

(73) Assignee: Molex, LLC, Lisle, IL (US)

(21) Appl. No.: 16/822,026

(22) Filed: Mar. 18, 2020

(30) Foreign Application Priority Data
Mar. 25, 2019 (JP) 2019-056447

(51) Int. Cl. A61M 25/09; A61B 5/0428; A61B 5/0478; H01R 13/33; H01R 24/58

(52) U.S. Cl. CPC A61M 25/09; A61B 5/04286; A61B 5/0478; H01R 2201/12; H01R 24/58; A61B 2562/225; H01R 13/33

(57) ABSTRACT

A connector is provided that has multiple electrode rings which are arranged in a row and capable of allowing multiple wires to pass therein The connector also has a relay ring capable of allowing the multiple wires to pass therein. The relay ring has a first mating part fitted to one electrode ring of two adjacent electrode rings and a second mating part fitted to the other electrode ring.

### RELATED APPLICATION

This application claims priority to Japanese Application Serial No. 2019-056447, filed on Mar. 25, 2019, which is incorporated by reference in its entirety.

### TECHNICAL FIELD

The present disclosure relates to the structure of a connector provided in a guidewire.

### BACKGROUND ART

One example of a guidewire utilized in catheter treatment is a guidewire having a sensor for detecting blood information (for example, blood pressure) at the wire tip. Blood information detected through the sensor is input to a monitor through a signal cable. The guidewire has a connector at the base end thereof, while the connector is connected to a counterpart connector present at the tip of the signal cable.

A guidewire disclosed in Patent Document 1 has multiple wires extending from the sensor serving as the tip. The connector has multiple electrode rings arranged in a row. Multiple electrode rings are respectively electrically connected to multiple wires. The electrode rings are connected to the wires by soldering.

Patent Document 1: JP 2004-255204 A

### SUMMARY

In the connector disclosed in Patent Document 1, multiple electrode rings are divided and disposed. Consequently, the connector tends to be deformed at the boundary between two adjacent electrode rings. When the connector tends to be deformed, for example, the connector and a counterpart connector are problematically not correctly connected.

The connector proposed in the present disclosure includes: multiple electrode rings including a first electrode ring and a second electrode ring which are arranged in a row and capable of allowing multiple wires to pass therein; and a relay ring capable of allowing the multiple wires to pass therein.

The relay ring includes a first mating part fitted to the first electrode ring, along with a second mating part fitted to the second electrode ring. The relay ring is electrically connected to one of the first electrode ring or the second electrode ring and insulated from the other thereof.

The guidewire proposed in the present disclosure includes the connector and multiple wires.

The connector and the guidewire can increase the strength of the connector via the relay ring.

A method for manufacturing the guidewire proposed in the present disclosure includes the steps of: fitting a first mating part of a first relay ring and a first electrode ring so as to form a first electrode ring assembly; fitting the first mating part of a second relay ring and a second electrode ring so as to form a second electrode ring assembly; fitting a second mating part of the first electrode ring assembly and the second electrode ring of the second electrode ring assembly; allowing a first wire and a second wire to pass through the first electrode ring, the first relay ring, the second electrode ring, and the second relay ring; and respectively connecting the first electrode ring and the second electrode ring to the first wire and the second wire.

This manufacturing method can increase the strength of the connector via the relay ring.

### BRIEF DESCRIPTION OF DRAWINGS

FIG. 1 is a view illustrating one example of a guidewire proposed in the present disclosure.

FIG. 2A is a perspective view illustrating one example of a connector possessed by the guidewire proposed in the present disclosure.

FIG. 2B is an exploded perspective view of the connector.

FIG. 3 is an exploded perspective view of an electrode ring assembly configuring the connector.

FIG. 4A is a cross sectional view of a guidewire.

FIG. 4B is a cross sectional view of a guidewire.

FIG. 5 is a cross sectional view of a guidewire. The cut surface is the surface indicated by line V-V in FIG. 4A.

FIG. 6A is a view describing a method for manufacturing a guidewire.

FIG. 6B is a view describing a method for manufacturing a guidewire.

FIG. 6C is a view describing a method for manufacturing a guidewire.

FIG. 7A is a perspective view illustrating a modified example of a connector.

FIG. 7B is a perspective view illustrating a modified example of a connector.

FIG. 8 is a cross sectional view of a guidewire having the connector illustrated in FIGS. 7A and 7B.

### DETAILED DESCRIPTION OF PREFERRED EMBODIMENTS

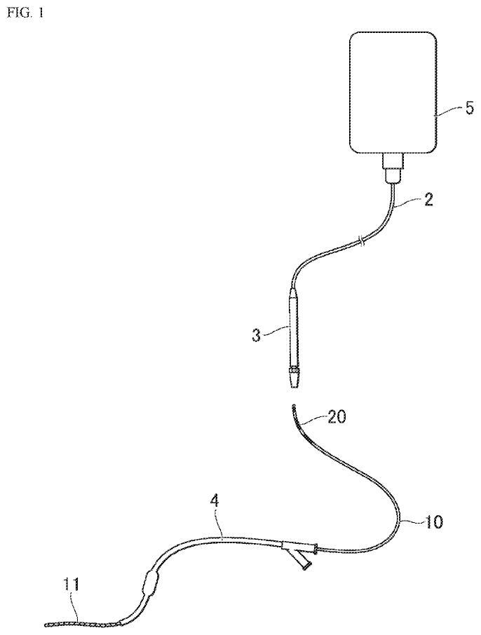

Hereinafter, an example of a guidewire and a connector proposed in the present disclosure will be described. The present specification describes a guidewire 10 and a connector 20 illustrated in FIG. 1, etc. as one example of a guidewire and a connector. In the following description, the direction indicated by Y1-Y2 of FIG. 1 is referred to as the “wire extending direction.”

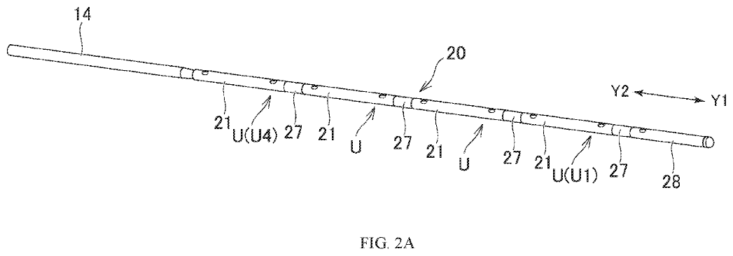

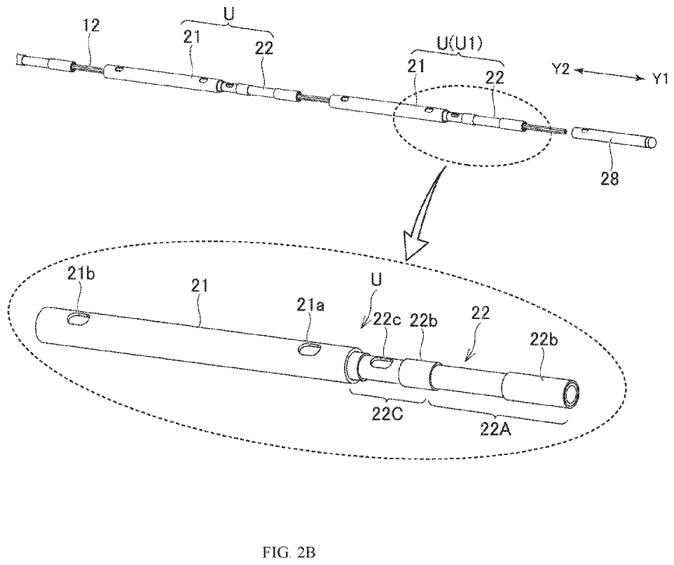

As illustrated in FIG. 1, during use, a guidewire 10 is inserted into a catheter 4. A sensor 11 is attached to the tip of the guidewire 10. A connector 20 may be provided at the base end as the distal end of the guidewire 10. An electric cable 2 is connected to a monitor 5, while the connector 20 is connected to a counterpart connector 3 provided at the tip of the electric cable 2. When using the guidewire 10, the tip, as the proximal end of the guidewire 10, is inserted into the body of a patient. When the guidewire 10 reaches the target position, the catheter 4 is guided by the guidewire 10. An output signal of the sensor 11 is input to the monitor 5 through wires 12 (see FIG. 2B) and the electric cable 2 as electric cables possessed by the guidewire 10.

As illustrated in FIGS. 2A and 2B, the connector 20 may have multiple electrode ring assemblies U arranged in a row in the wire extending direction. Multiple wires 12 pass through the inside of the electrode ring assemblies U. In one example, the guidewire 10 has four wires 12, while the connector 20 has four electrode ring assemblies U. The number of the wires 12 may be less than four or more than four. The connector 20 may further have a tip electrode ring 28. The tip electrode ring 28 is disposed at the base end of the guidewire 10 (the tip of the connector 20).

Each electrode ring assembly U may be configured by an electrode ring 21 and a relay ring 22. As illustrated in FIG. 2B, the electrode ring 21 is elongated and cylindrical in the wire extending direction, with multiple wires 12 capable of passing through the inside thereof. The cross section of the electrode ring 21 may be circular so as to surround the entire periphery of the multiple wires 12. As described later in detail, multiple electrode rings 21 are respectively electrically connected to the multiple wires 12. When the connector 20 is connected to the counterpart connector 3, the terminal of the counterpart connector 3 contacts the outer peripheral surface of each electrode ring 21.

The electrode ring 21 is, for example, formed of metals such as stainless steel and copper. The outer peripheral surface of the electrode ring 21 may be plated (for example, gold plated). The diameter of the outer peripheral surface of the electrode ring 21 may be, for example, 0.2 mm to 0.6 mm. The diameter of the outer peripheral surface of the electrode ring 21 may be uniform from one end to the other end of the electrode ring 21.

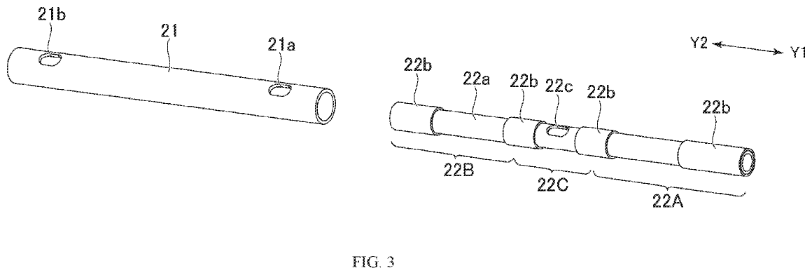

As illustrated in FIG. 3, the relay ring 22 is also elongated and cylindrical in the wire extending direction, with multiple wires 12 capable of passing through the inside thereof. As with the electrode ring 21, the cross section of the relay ring 22 may be circular so as to surround the entire periphery of the multiple wires 12.

When the relay ring 22 is fitted to the electrode ring 21, the relay ring 22 has a part fitted to the electrode ring 21 configuring the same electrode ring assembly U, along with a part protruding from the electrode ring 21 in the wire extending direction. Hereinafter, the part protruding from the electrode ring 21 is referred to as a “first mating part 22A,” while the part mating with the electrode ring 21 configuring the same electrode ring assembly U is referred to as a “second mating part 22B.” The relay ring 22 may have a central part 22C between two mating parts 22A, 22B. As mentioned later, the central part 22C is a part fitted to neither of two adjacent electrode rings 21. The multiple wires 12 can pass through the two mating parts 22A, 22B and the central part 22C.

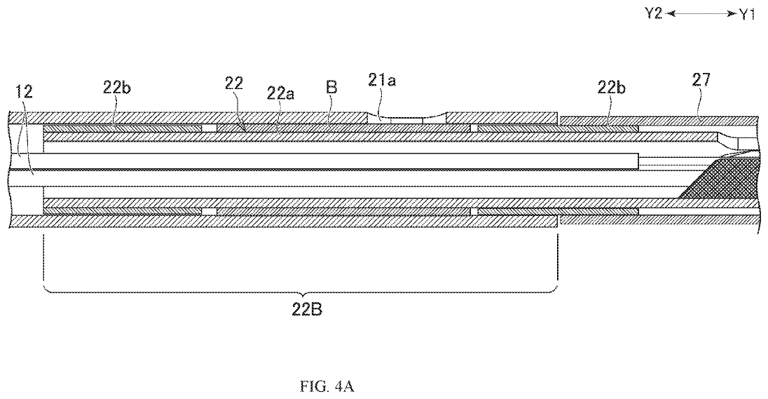

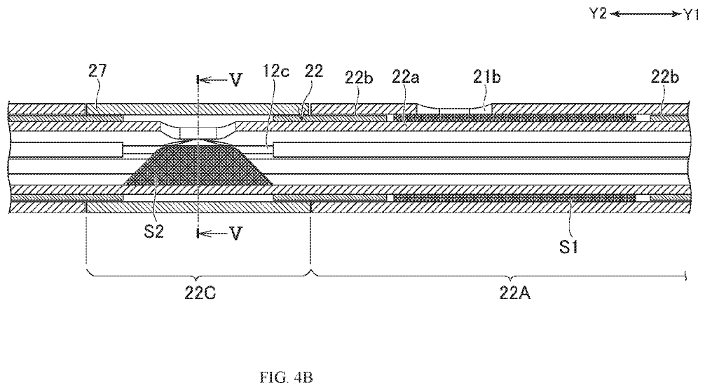

The second mating part 22B is fitted to the inside of the electrode ring 21. The first mating part 22A is fitted to the inside of the electrode ring 21 of an adjacent electrode ring assembly U (see FIG. 4B). That is, the first mating part 22A of the relay ring 22 is fitted to one electrode ring 21 of two adjacent electrode rings 21, while the second mating part 22B is fitted to the other electrode ring 21. Consequently, the relay ring 22 can prevent the connector 20 from bending at the boundary between two adjacent electrode rings 21. That is, it is possible to increase the strength of the connector 20.

As mentioned above, the cross section of the relay ring 22 is circular so as to surround the entire periphery (360°) of the multiple wires 12. The shape of the relay ring 22 is not limited to this. The cross section of the relay ring 22 may be arc shaped so as to surround the wires 12. For example, the cross section of the relay ring 22 may be arc shaped so as to surround the wires 12 in the angle range of 270° or greater. Such a structure can sufficiently ensure the strength of the relay ring 22 when it comes to the bending of the connector 20.

Two relay rings 22 are fitted to one electrode ring 21. That is, the first mating part 22A of one relay ring 22, along with the second mating part 22B of the other relay ring 22, is fitted to the inside of one electrode ring 21. The length (L1) of the first mating part 22A fitted to the inside of the electrode ring 21 is, for example, larger than one fourth the length (L3) of the electrode ring 21 (L1>L3/4). Similarly, the length (L2) of the second mating part 22B fitted to the inside of the electrode ring 21 is, for example, larger than one fourth the length (L3) of the electrode ring 21 (L2>L3/4). In this manner, because the length of the part fitted to the electrode ring 21 is long, deformation of the connector 20 can be further effectively suppressed.

Note that the length (L1) of the first mating part 22A may be larger than one third the length (L3) of the electrode ring 21 (L1>L3/3). Similarly, the length (L2) of the second mating part 22B may be larger than one third the length (L3) of the electrode ring 21 (L2>L3/3). The total of the length (L1) of the first mating part 22A and the length (L2) of the second mating part 22B may be larger than half the length (L3) of one electrode ring 21 (that is, (L1+L2)>L3/2). The total of the length (L1) of the first mating part 22A and the length (L2) of the second mating part 22B may be larger than two thirds the length (L3) of one electrode ring 21 (that is, (L1+L2)>L3×⅔).

Moreover, the total length (L1+L2) of the first mating part 22A and the second mating part 22B of the relay ring 22 is larger than the length L4 of the central part 22C. Accordingly, because most of the relay ring 22 is inserted into the electrode ring 21, the connector 20 can be effectively prevented from bending.

In one electrode ring assembly U, the center position of the electrode ring 21 coincides with the center position of the relay ring 22 (second mating part 22B). (Here, the “center position” is the center position of rings 21, 22 in the radial direction.) Consequently, when multiple electrode rings 21 are coupled via the relay ring 22, these are disposed in the same straight line.

The electrode ring 21 and the relay ring 22 configuring one electrode ring assembly U may be fixed to each other. Moreover, these two rings 21, 22 may be electrically insulated. The relay ring 22 (second mating part 22B) and the electrode ring 21 have, for example, the following fixing structure.

As illustrated in FIG. 3, the relay ring 22 may have a ring core 22a. The ring core 22a is elongated and cylindrical in the wire extending direction. The ring core 22a has a part configuring the first mating part 22A, a part configuring the second mating part 22B, and a part configuring the central part 22C. The ring core 22a may be formed by a conductor. Moreover, the material of the ring core 22a may be different from the material of the electrode ring 21. The material of the ring core 22a is, for example, a metal such as nickel or copper. The material of the ring core 22a may be the same as the material of the electrode ring 21.

As illustrated in FIG. 3, the relay ring 22 may have multiple insulating spacers 22b (formed by an insulating material) on the outer peripheral surface of the ring core 22a. The material of the insulating spacers 22b is, for example, ceramic, but is not limited to this as long as it is a material having insulation. Portions of the multiple insulating spacers 22b are disposed in the second mating part 22B. As illustrated in FIG. 4A, the insulating spacers 22b, along with the ring core 22a, are fitted to the inside of the electrode ring 21, thereby ensuring a gap (space) between the outer peripheral surface of the ring core 22a and the inner peripheral surface of the electrode ring 21. The inner peripheral surface of the electrode ring 21 and the second mating part 22B are fixed to each other via an adhesive B provided in this gap.

An opening 21a (see FIG. 2B) may be formed in the electrode ring 21. As illustrated in FIG. 4A, the opening 21a corresponds to the position of the gap formed by the abovementioned insulating spacers 22b. A portion of the outer peripheral surface of the relay ring 22 (second mating part 22B) is disposed inside the opening 21a in the radial direction of the electrode ring 21. The opening 21a penetrates through the electrode ring 21, wherein the outer peripheral surface of the second mating part 22B (the outer peripheral surface of the ring core 22a) is exposed via the opening 21a. In the manufacturing process of a guidewire 10, the adhesive B is provided in the gap between the outer peripheral surface of the second mating part 22B and the inner peripheral surface of the electrode ring 21 via the opening 21a. Therefore, at least a portion of the adhesive B is visible through the opening 21a and present along the periphery of the edge of the opening 21a.

Moreover, the adhesive B may be formed along the entire periphery of the relay ring 22. Accordingly, the relay ring 22 and the electrode ring 21 can be adhered to each other over a wide range so as to ensure the fixing strength between the relay ring 22 and the electrode ring 21.

For example, adhesives of photocurable resin, thermosetting resin, normal temperature curable resin, two-pack curable resin, etc. can be utilized as an adhesive B. However, any material may be used as long as it is a material which is a resin having insulation and which fixes the relay ring 22 and the electrode ring 21 to each other.

As illustrated in FIG. 3, the insulating spacers 22b is circular so as to surround the ring core 22a. The thickness of the insulating spacers 22b may be substantially uniform along the entire periphery of the relay ring 22. In so doing, the thickness of the adhesive B is substantially uniform along the entire periphery of the relay ring 22. Consequently, the center position of the electrode ring 21 coincides with the center position of the relay ring 22 (second mating part 22B).

Moreover, as illustrated in FIG. 4A, the second mating part 22B has insulating spacers 22b at two positions separated in the wire extending direction, wherein both of these two insulating spacers 22b may be fitted to the inside of the electrode ring 21. In so doing, inclination of the relay ring 22 to the electrode ring 21 is suppressed. The two insulating spacers 22b are, for example, formed at both ends of the second mating part 22B. The opening 21a for supplying the adhesive B is disposed between the two insulating spacers 22b.

Multiple electrode rings 21 are respectively electrically connected to multiple wires 12. For example, the electrode rings 21 may be electrically connected to the relay ring 22, while the relay ring 22 may be electrically connected to the wires 12. That is, the electrode rings 21 may be indirectly connected to the wires 12.

An example of the connection structure between the relay ring 22 and the adjacent electrode ring 21 will be described. (An “adjacent electrode ring” refers to an electrode ring configuring an adjacent electrode ring assembly U.) As illustrated in FIG. 4B, the relay ring 22 and the adjacent electrode ring 21 are electrically connected and fixed to each other via a conductive material Si filled into the space (gap) between the outer peripheral surface of the first mating part 22A and the inner peripheral surface of the adjacent electrode ring 21. The conductive material Si is, for example, a conductive adhesive, solder, etc.

As illustrated in FIG. 4B, an opening 21b is formed in the electrode ring 21. The opening 21b corresponds to the position of the outer peripheral surface of the first mating part 22A. A portion of the outer peripheral surface of the relay ring 22 (first mating part 22A) is disposed inside the opening 21b in the radial direction of the electrode ring 21. When the opening 21b penetrates through the electrode ring 21, and the first mating part 22A is fitted to the inside of the adjacent electrode ring 21 in the manufacturing process of a connector 20, the outer peripheral surface of the first mating part 22A (the outer peripheral surface of the ring core 22a) is exposed via the opening 21b. The conductive material S1 may be supplied via the opening 21b. In this case, at least a portion of the conductive material S1 is visible through the opening 21b and present along the periphery of the edge of the opening 21b.

The conductive material S1 may be formed along the entire periphery of the relay ring 22. Consequently, the relay ring 22 and the electrode ring 21 are electrically connected to each other over a wide range. As a result, a strong and electrically reliable connection can be ensured between the relay ring 22 and the electrode ring 21.

The insulating spacers 22b are formed along the entire periphery of the relay ring 22 in a substantially uniform thickness. Therefore, the thickness of the conductive material S1 is also substantially uniform along the entire periphery of the relay ring 22. Consequently, the center position of the adjacent electrode ring 21 can precisely coincide with the center position of the relay ring 22 (first mating part 22A).

Moreover, as illustrated in FIG. 3, the first mating part 22A has insulating spacers 22b at two positions separated in the wire extending direction, wherein both of these two insulating spacers 22b may be fitted to the inside of the adjacent electrode ring 21. In so doing, the inclination of the relay ring 22 to the adjacent electrode ring 21 is suppressed. The two insulating spacers 22b are, for example, formed at both ends of the first mating part 22A. When the first mating part 22A is fitted to the adjacent electrode ring 21, the opening 21b for supplying the conductive material S1 is disposed between the two insulating spacers 22b. As mentioned above, because the inclination of the relay ring 22 is suppressed and the conductive material S1 is provided in a uniform gap between the first mating part 22A of the relay ring 22 and the electrode ring 21 via the opening 21b, the center position of the electrode ring 21 can further precisely coincide with the center position of the relay ring 22.

Note that the connection structure between the relay ring 22 and the adjacent electrode ring 21 is not limited to the example of the connector 20. For example, the relay ring 22 and the adjacent electrode ring 21 may be fixed to each other and electrically connected by welding. In this case, in order to weld the relay ring 22 and the adjacent electrode ring 21, for example, laser welding can be utilized.

Moreover, the structure of the relay ring 22 is not limited to that of the example of the connector 20. For example, the outer peripheral surface of the ring core 22a may be collectively covered with an insulating material. In addition, the ring core 22a may be exposed only near the opening 21a, 21b of the electrode ring 21, while the adhesive B and the conductive material S1 may be respectively supplied thereto.

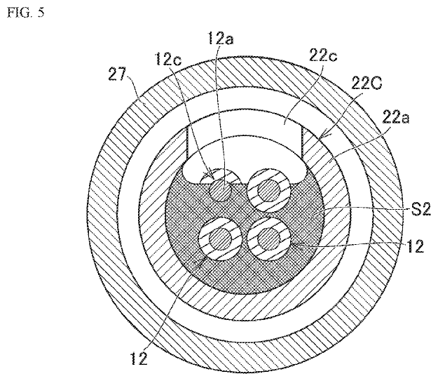

The connection structure between the relay ring 22 and the wires 12 will hereinafter be described. As illustrated in FIGS. 4B and 5, the wires 12 have a core wire 12a (formed by a conductor), along with an outer sheath 12b covering the core wire 12a. The wires 12 have a strip part 12c on which the core wire 12a (with the outer sheath 12b peeled) is exposed. Each wire 12 has one strip part 12c. The position of the strip part 12c of multiple wires 12 respectively corresponds to the position of the central part 22C (see FIG. 2B) of multiple relay rings 22. The inner peripheral surface of the central part 22C and the strip part 12c of the wires 12 are electrically connected via a conductive material S2 provided inside the central part 22C.

As illustrated in FIGS. 4B and 5, an opening 22c is formed in the relay ring 22 (more specifically, the central part 22C). Multiple wires 12 are disposed inside the opening 22c in the radial direction of the relay ring 22. The opening 22c corresponds to the position of the strip part 12c of one wire 12, wherein the strip part 12c is disposed inside the opening 22c in the radial direction of the relay ring 22. The opening 22c penetrates through the relay ring 22, wherein, in the manufacturing process of a guidewire 10, the strip part 12c is exposed via the opening 22c. The conductive material S2 is provided inside the relay ring 22 via the opening 22c. Moreover, when solder is utilized as a conductive member, the inner peripheral surface of the relay ring 22 may be plated (for example, gold plated). As a result, the electric connection stability between the core wire 12a and the relay ring 22 can be improved.

According to the structure of the guidewire 10 in which the electrode ring 21 and wires 12 are electrically connected via the relay ring 22, that is, according to the structure in which the relay ring 22 and a conductor 12 are directly electrically connected, in the state in which the connector 20 mates with a counterpart connector, the electrode ring 21 receives electric contact force with the counterpart connector, but does not receive the load of the relay ring 22. Therefore, because a load is not applied to the connecting part between the relay ring 22 and the wire 12, the relay ring 22 and the wires 12 can be prevented from being disconnected.

As illustrated in FIGS. 2A and 5, a tube 27 covering the opening 22c of the relay ring 22 may be fitted to the central part 22C of the relay ring 22. As the tube 27, for example, a heat shrinkable tube which shrinks by heating can be utilized. This tube 27 can reduce protrusions and recesses on the outer surface of the connector 20.

Note that, unlike the example of the guidewire 10, the electrode ring 21 may be directly connected to the wires 12. For example, the relay ring 22 may have an opening at the position of the opening 21b of the electrode ring 21 so as to expose the inner surface of the electrode ring 21. The wires 12 may have the strip part 12c at the position of this opening. In addition, a conductive material may be provided to a part on which the inner surface of the electrode ring 21 is exposed, as well as to the strip part 12c. In so doing, the electrode ring 21 is electrically connected to the wire 12 not via the relay ring 22.

As mentioned above, the connector 20 has a tip electrode ring 28 at the tip thereof. The tip electrode ring 28 is cylindrical with the tip closed. The tip electrode ring 28 mates with the first mating part 22A of the relay ring 22 of an electrode ring assembly U (FIG. 2B the symbol U1) closest to the tip.

The tip electrode ring 28 may be electrically connected to the relay ring 22. The connection structure thereof may be, for example, the same as that illustrated in the example of FIG. 4B. That is, the inner peripheral surface of the tip electrode ring 28 may be connected to the outer peripheral surface of the first mating part 22A of a conductive material the relay ring 22. An opening for supplying this conductive material may be formed in the tip electrode ring 28.

As mentioned above, the guidewire 10 has four wires 12. In contrast, the connector 20 has four electrode rings 21 along with one tip electrode ring 28. Therefore, one of the four electrode rings 21 does not have to be electrically connected to wires 12. For example, the electrode rings 21 of the electrode ring assembly U (symbol U4 of FIG. 2A) disposed on the opposite side of the tip electrode ring 28 may not be connected to wires 12.

As illustrated in FIG. 2A, the guidewire 10 has a shaft 14 through which multiple wires 12 pass. The electrode ring 21 of the electrode ring assembly U4 disposed on the opposite side of the tip electrode ring 28 may mate with the shaft 14.

Examples of the method for manufacturing the guidewire 10 will be described.

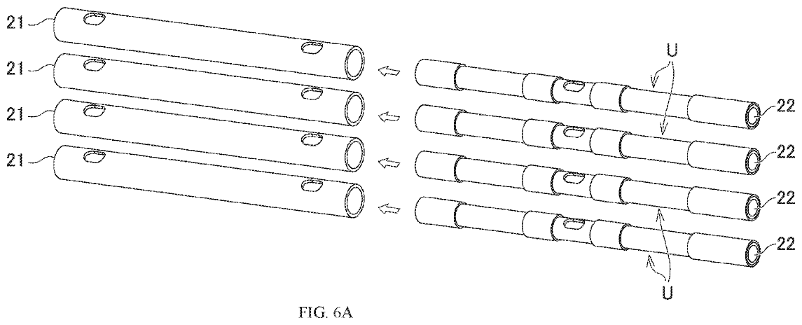

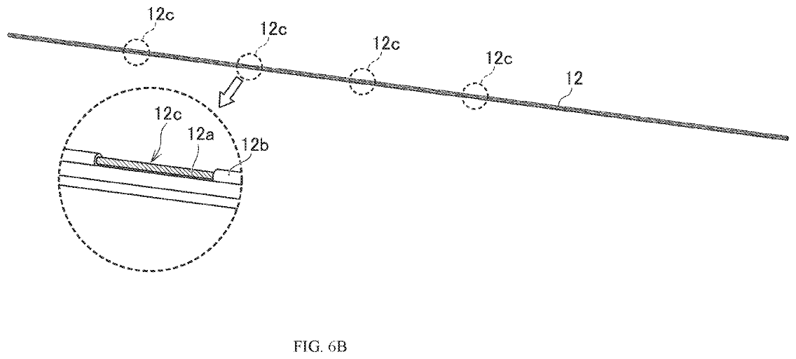

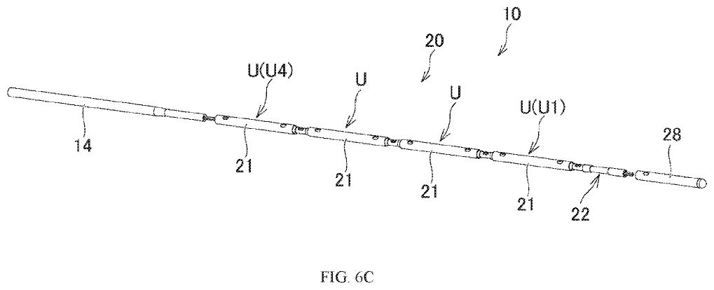

As illustrated in FIG. 6A, multiple relay rings 22 respectively mate with multiple electrode rings 21. In addition, an adhesive B is supplied from an opening 21a (see FIG. 2B) of each electrode ring 21 so as to fix the relay ring 22 and the electrode ring 21. As a result, multiple electrode ring assemblies U are obtained. The adhesive B may be any adhesive. If the adhesive B is photocurable, for example, ultraviolet curable resin is utilized, allowing multiple electrode ring assemblies U to be simultaneously irradiated with ultraviolet rays. While rings 21, 22 configuring four electrode ring assemblies U are illustrated in FIG. 6A, the number of electrode ring assemblies U which are simultaneously irradiated with ultraviolet rays may be more than four.

As illustrated in FIG. 6B, by peeling the outer sheath 12b of multiple wires 12, the strip part 12c is formed in each wire 12. The positions of the strip parts 12c respectively correspond to the positions of multiple relay rings 22.

Next, as illustrated in FIG. 6C, multiple electrode ring assemblies U are sequentially inserted into the wire 12. In addition, the relay ring 22(first mating part 22A) mates with the adjacent electrode ring 21. At this time, the central part 22C of the relay ring 22 is exposed between two electrode rings 21. In addition, a conductive material (for example, a conductive adhesive, thread solder, etc.) is supplied from an opening 21b (see FIG. 4B) of each electrode ring 21, allowing the relay ring 22 and the electrode ring 21 to be electrically connected and fixed to each other. Moreover, a conductive material (for example, a conductive adhesive, thread solder, etc.) is supplied via an opening 22c (see FIG. 4B) formed in the central part 22C of the relay ring 22 so as to electrically connect the strip part 12c of the wire 12 and the relay ring 22.

The tip electrode ring 28 (see FIG. 2B) mates with the relay ring 22 of an electrode ring assembly U1 disposed at the tip thereof. The tip electrode ring 28 is also connected to the relay ring 22 via a conductive material. Moreover, the shaft 14 (see FIG. 2A) is fitted to the inside of the electrode ring 21 of an electrode ring assembly U4 disposed at the rear end. The shaft 14 and the electrode ring 21 are also fixed via the adhesive. Lastly, a tube 27 is fitted to the central part 22C of the relay ring 22 so as to cover the opening 22c of the relay ring 22. As the tube 27, the abovementioned heat shrinkable tube can be utilized.

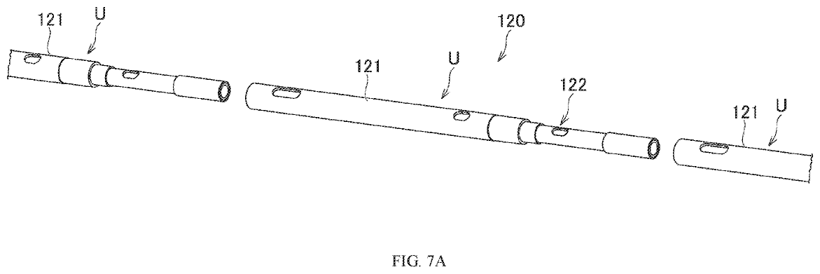

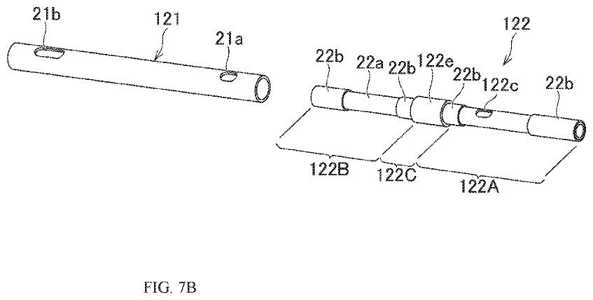

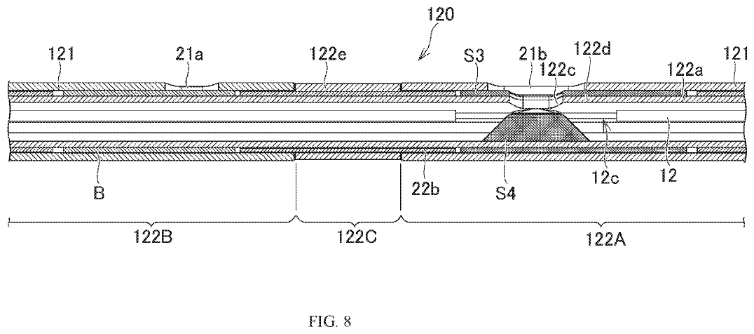

FIGS. 7A, 7B, and 8 are views each illustrating a connector 120, which is a modified example of the connector 20 proposed in the present disclosure. In these figures, the same elements as the thus described elements (components, members, and regions) are labeled with the same symbols. Hereinafter, differences between the connector 20 and the connector 120 will be mainly described. The same structure as that of the connector 20 may be applied to the items with no explanation thereof

As illustrated in FIG. 7B, each electrode ring assembly U has an electrode ring 121 and a relay ring 122. As with the electrode ring 21 illustrated in FIG. 3, the electrode ring 121 has the openings 21a, 21b. In contrast, the relay ring 122 has a first mating part 122A, a second mating part 122B, and a central part 122C.

In one electrode ring assembly U, the electrode ring 121 and the relay ring 122 are electrically insulated and fixed to each other. As illustrated in FIG. 8, the position of the opening 21a of the electrode ring 121 corresponds to the position of a ring core 22a in the second mating part 122B. The relay ring 122 and the electrode ring 121 are fixed via the adhesive B which is provided between the outer peripheral surface of the second mating part 122B and the inner peripheral surface of the electrode ring 121 via the opening 21a. Multiple insulating spacers 22b are formed on the outer peripheral surface of the relay ring 122.

An opening 122c (see FIG. 7B) is formed in the first mating part 122A of the relay ring 122. As illustrated in FIG. 8, when two adjacent electrode ring assemblies U are coupled, the position of the opening 122c of the relay ring 122 of one assembly U coincides with the position of the opening 21b of the electrode ring 121 of the other assembly U. The wires 12 are disposed inside two openings 21b, 122c in the radial direction of rings 122, 121, such that the wires 12 are exposed through the two openings 21b, 122c. One of multiple wires 12 has the strip part 12c at the positions of these openings 21b, 122c.

As illustrated in FIG. 8, the electrode ring 121 and the relay ring 122 are electrically connected and fixed to each other via a conductive material S3 which is supplied between the outer peripheral surface of the relay ring 122 and the inner peripheral surface of the electrode ring 121 via the opening 21b of the electrode ring 121. Moreover, the strip part 12c of wires 12 and the inner peripheral surface of the relay ring 122 are electrically connected via a conductive material S4 which is provided inside the relay ring 122 via the opening 21b and the opening 122c of the relay ring 122. In this manner, because the positions of the two openings 21b, 122c coincide with each other, the operation of attaching a conductive material can be facilitated.

Note that the size of the opening 21b formed in the electrode ring 121 is larger than the opening 122c of the relay ring 122. Consequently, a portion 122d (the vicinity of the edge of the opening 122c, see FIG. 8) of the outer peripheral surface of the relay ring 122 is exposed via the opening 21b. Consequently, the operation of supplying the conductive material S3 between the outer peripheral surface of the relay ring 122 and the inner peripheral surface of the electrode ring 121 can be facilitated.

The relay ring 122 has insulating spacers 122e (see FIG. 7B) at the central part 122C. The insulating spacers 122e is disposed between two adjacent electrode rings 121 so as to electrically insulate these. The outer peripheral surface of insulating spacers 122d is substantially flush with the outer peripheral surface of the electrode ring 121.

As described above, the connector proposed in the present disclosure has: multiple electrode rings 21 which are arranged in a row and capable of allowing multiple wires to pass therein; and multiple relay ring 22 capable of allowing the multiple wires 12 to pass therein. Each of multiple relay rings 22 has: a first mating part 22A fitted to one electrode ring 21 of two adjacent electrode rings 21; and a second mating part 22B fitted to the other electrode ring 21. The connectors 20, 120 and guidewires 10, 110 can increase the strength of the connectors 20, 120 via the relay ring 22. That is, the connector can be prevented from bending at the boundary between two electrode rings 21.

The connector and guidewire proposed in the present disclosure are not limited to the abovementioned connectors 20 and 120.

Unlike the connectors 20, 120, an electrode ring 21 and the relay ring 22 configuring one electrode ring assembly U may be electrically connected. For example, a conductive material may be supplied via an opening 21a (see FIG. 2B) of the electrode ring 21, such that the electrode ring 21 and the relay ring 22 may be electrically connected and fixed to each other. In addition, the wires 12 and the relay ring 22 may be electrically connected via a conductive material supplied from the opening 22c (FIG. 2B) of the relay ring 22. As another example, the relay ring 22 and the electrode ring 21 may be fixed to each other and electrically connected by welding (for example, laser welding). If welding is used to connect the relay ring 22 and the electrode ring 21, the connector 20 may not have insulating spacers 22b between the relay ring 22 and the electrode ring 21 configuring one electrode ring assembly U.

Moreover, in the structure in which the electrode ring 21 and the relay ring 22 configuring one electrode ring assembly U are electrically connected, the first mating part 22A of the relay ring 22 may mate with the adjacent electrode ring 21 so as to be adhered thereto via an adhesive (that is, insulate two rings 21, 22). As still another example, the first mating part 22A of the relay ring 22 mates with the adjacent electrode ring 21, so the two rings 21, 22 do not have to be fixed via the adhesive.

1. A connector provided in a guidewire, comprising:
multiple electrode rings including a first electrode ring and a second electrode ring which are arranged in a row and capable of allowing multiple wires to pass therein; and
a relay ring capable of allowing the multiple wires to pass therein,
wherein the relay ring includes a first mating part fitted to the first electrode ring, along with a second mating part fitted to the second electrode ring, and
wherein the relay ring is electrically connected to one of the first electrode ring or the second electrode ring and insulated from the other thereof

2. The connector according to claim 1, wherein the relay ring further includes a central part disposed between the first mating part and the second mating part, and
wherein the total length of the first mating part and the second mating part of the relay ring is larger than the length of the central part.

3. The connector according to claim 1, wherein an opening is formed in one electrode ring of the first electrode ring and the second electrode ring, and
wherein the outer peripheral surface of the relay ring has a part disposed inside the opening of the one electrode ring in the radial direction of the electrode ring.

4. The connector according to claim 3, wherein the electrode ring and the relay ring are fixed to each other via an adhesive or a conductive material which is disposed between the inner peripheral surface of the one electrode ring and the outer peripheral surface of the relay ring, and
wherein at least a portion of the adhesive or at least a portion of the conductive material is visible through the opening of the one electrode ring,

5. The connector according to claim 1, wherein an opening is formed in at least one ring of the relay ring, the first electrode ring, and the second electrode ring, and
wherein the multiple wires are disposed inside the opening in the radial direction of the at least one ring.

6. A guidewire, comprising: the connector according to claim 1; and the multiple wires.

7. The guidewire according to claim 6, wherein an opening is formed in at least one ring of the relay ring, the first electrode ring, and the second electrode ring, and
wherein the multiple wires are disposed inside the opening in the radial direction of the at least one ring, and
wherein the at least one ring and one wire of the multiple wires are connected to each other via a conductive material visible through the opening.

8. The guidewire according to claim 7, wherein one electrode ring of the first electrode ring and the second electrode ring is electrically connected to one of the multiple wires via the relay ring.

9. A method for manufacturing a guidewire, comprising the steps of: fitting a first mating part of a first relay ring and a first electrode ring so as to form a first electrode ring assembly;
fitting the first mating part of a second relay ring and a second electrode ring so as to form a second electrode ring assembly;
fitting a second mating part of the first electrode ring assembly and the second electrode ring of the second electrode ring assembly;
allowing a first wire and a second wire to pass through the first electrode ring, the first relay ring, the second electrode ring, and the second relay ring; and
respectively connecting the first electrode ring and the second electrode ring to the first wire and the second wire.

10. The method for manufacturing a guidewire according to claim 9, wherein a conductive material is provided to an opening formed in one ring of the first relay ring and the first electrode ring, and
wherein the first wire and the one ring are electrically connected.

11. The method for manufacturing a guidewire according to claim 9, wherein a conductive material is supplied between the first electrode ring and the first relay ring via an opening formed in the first electrode ring.
